(19) United States
(12) Patent Application Publication
(10) Pub. No.: US 2015/0032051 A1
(43) Pub. Date: Jan. 29, 2015

(54) VENTED REFILL ARRANGEMENT FOR IMPLANTABLE DRUG-DELIVERY DEVICES

(71) Applicants: William Andrew Brandt, Castaic, CA (US); Jilian Kavazov, Arcadia, CA (US); David Mathew Wessel, Sunland, CA (US); Andrew Dunn, Santa Monica, CA (US)

(72) Inventors: William Andrew Brandt, Castaic, CA (US); Jilian Kavazov, Arcadia, CA (US); David Mathew Wessel, Sunland, CA (US); Andrew Dunn, Santa Monica, CA (US)

(21) Appl. No.: 14/317,848

(22) Filed: Jun. 27, 2014

Related U.S. Application Data
(60) Provisional application No. 61/840,070, filed on Jun. 27, 2013.

(51) Int. Cl. A61M 39/02; A61M 39/04

(52) U.S. Cl. CPC A61M 39/0247; A61M 39/04; A61M 2039/027; A61M 2039/0276; A61M 2039/0258; A61M 2209/045; A61M 2205/75
USPC 604/143

(57) ABSTRACT

Embodiments of the present invention provide a vent arrangement integrated with a refill port disposed on the outer shell of an implantable drug-delivery device. The vent arrangement may utilize a tiered structure with two septums in a space-efficient configuration that facilitates both venting of pressure and refill of the drug reservoir.

### CROSS-REFERENCES TO RELATED APPLICATIONS

This application claims priority to and the benefit of U.S. Provisional Patent Application No. 61/840,070, filed on Jun. 27, 2013, the entire disclosure of which is hereby incorporated herein by reference.

### BACKGROUND

Most drug-delivery devices utilize an actuation mechanism to drive medicament from a reservoir through a cannula into target areas. In general, pressurization occurs within the drug-delivery device or at the interface between the device and its surroundings. The pressure magnitudes and gradients in these regions can make it difficult to precisely control delivery of small amount of drugs, especially when the device is refillable or used for repeated dosing over a relatively long term. For example, without proper regulation of the pressure in the drug reservoir, pressure or vacuum buildup can interfere with smooth, continuous administration of a liquid medicament. This problem is particularly challenging in the devices whose driving mechanism involves generation of pressurized gas; in such devices, generated gas may leak to various device regions. In addition, when the device is implanted in a human body, the difficulties of limited physical space and access to the device, as well as the overall complexity of in vivo implantation and operation, can make pressure regulation in the device challenging.

Gas-driven drug-delivery devices may produce excess gas, and ensuring gas-tightness along the pressurization passage can require significant efforts in design, manufacture and quality control. For example, in electrolytic drug-delivery devices, hydrogen and oxygen are generated as an actuating mechanism during dosing. Hydrogen is known to penetrate thin walls easily and leak into reservoir chambers and their perimeters, resulting in inaccurate pressure-dosing characteristics or even unintended delivery of gas. For some drug-delivery regimes, instantaneous bursts of drug may be required (alone or to supplement steady-state delivery). The excess gas and its effects on delivery accuracy can pose major difficulties, especially in the sub-milliliter scale.

Excess gas can also adversely affect the refilling of drug-delivery devices. As the excess gas accumulates in the drug reservoir chambers, refill routes, and/or other adjacent chambers, it can complicate the refilling process and create considerable dead volume. More importantly, a variety of drug-delivery devices have compliant reservoir walls to minimize dead volumes and provide ease in handling during refilling. With these devices, the excess gas accumulating in the perimeter creates a differential pressure that can eventually prevent the refilling operation from proceeding to completion.

Venting may seem like an obvious solution to unwanted gas buildup, but can be difficult to achieve in devices intended for implantation. While valved passages connecting the pump to a portion outside of the body have been proposed for managing excess gas in drug-delivery devices, such an approach is unsuitable for biomedical implants as the transport of gases through the human body via a catheter or artificial vehicle for venting may be painful and increase risk of infection. In addition, as most biomedical implants are highly integrated and miniaturized, the limited physical space and access to the device further complicates venting: the venting component in an implantable drug-delivery device must generally be compact, easy to integrate and, notably, compatible with the human body environment in which various body fluids and tissues may interact with the vent.

### SUMMARY

Embodiments of the present invention provide a vent arrangement integrated with a refill port disposed on the outer shell of an implantable drug-delivery device. The vent arrangement may utilize a tiered structure with two septums in a space-efficient configuration that facilitates both venting of pressure and refill of the drug reservoir. Unlike septum configurations that utilize a movable septum with multiple positions (e.g., open and closed), the deflection of the septums in this configuration is minimal, allowing it to occupy less space within an implantable device. This configuration can also be used for other devices that may benefit from venting or pressure equilibration. All components of the refill port may be made of biocompatible materials and may additionally be translucent.

Accordingly, in a first aspect, the invention pertains to an implantable device for administering a liquid. In various embodiments, the device comprises an outer shell defining an interior volume; within the interior volume, a pump assembly including a reservoir, a gas-driven forcing mechanism and a passage for conducting liquid from the reservoir to an ejection site outside the shell in response to pressure applied by the forcing mechanism; and a refill port assembly that itself comprises (i) an orifice through a surface of the housing for receiving a refill needle; (ii) a first housing defining a first chamber having first and second open ends and fluidly coupled, via at least one bore through the first housing, to a ventable interior portion of the shell; (iii) a second housing defining a second chamber having an open end and fluidly coupled, via at least one bore through the second housing, to the drug reservoir; and (iv) first and second needle-penetrable septums, wherein the orifice, the first and second housings and the first and second septums are arranged in series with the first septum disposed between, and penetrably sealing, the orifice and the first open end of the first housing, and the second septum disposed between, and penetrably sealing, the second open end of the first housing and the open end of the second housing.

The first septum may be slit to form a check valve facilitating release of pressurized gas or relief of a vacuum within the ventable interior portion of the shell. In some embodiments, the first septum has a surface comprising an oleophobic coating thereover to discourage tissue ingrowth and endothelialization. The first septum may comprise or consist essentially of a polymeric material having a durometer ranging from 30 to 80. The second septum may be made of a self-healing material. In some embodiments, the bores are sized to function as a filter.

The second housing may comprise a closed end opposite the open end, where at least the closed end is made of a needle-impenetrable material. In some embodiments, the first and second housings and the first and second septums are received within separate recesses within the implantable device. The first septum may comprises a plurality of slits intersecting at a point. In some embodiments, the first and second septums further comprise one or more surface TEFLON layers and/or one or more surface layers of support mesh.

In some embodiments, each of the septums has first and second regions having, respectively, a first and second durometer; the first region includes at least a portion of an exterior of the associated septum, and the first durometer is higher than the second durometer. The first and second chambers may be filled with an open-cell material to provide structural support without sacrificing fluid flow.

In some embodiments, the fluid coupling between the first chamber and the ventable interior portion of the shell, and/or between the second chamber and the drug reservoir, comprises a polymer tube having an integrated check valve.

### BRIEF DESCRIPTION OF THE DRAWINGS

The foregoing will be more readily understood from the following detailed description of the invention, in particular, when taken in conjunction with the drawings, in which:

FIG. 1 is a side view of an implantable, refillable drug pump device in accordance with various embodiments of the invention.

FIG. 2 is a side view of the device shown in FIG. 1 deployed within an exterior housing.

FIGS. 3 and 4 are schematic and cutaway perspective views, respectively, of a refill structure in accordance with various embodiments of the invention.

FIGS. 5-9 are sectional views of various alternative embodiments of a refill structure in accordance herewith.

### DETAILED DESCRIPTION

The present invention relates, generally, to implantable drug pump devices with refillable drug reservoirs. Various embodiments described herein relate specifically to drug pump devices implanted into the eye (e.g., between the sclera and conjunctiva); however, many features relevant to such ophthalmic pumps are also applicable to other drug pump devices, such as, e.g., implantable insulin pumps, inner ear pumps, and brain pumps.

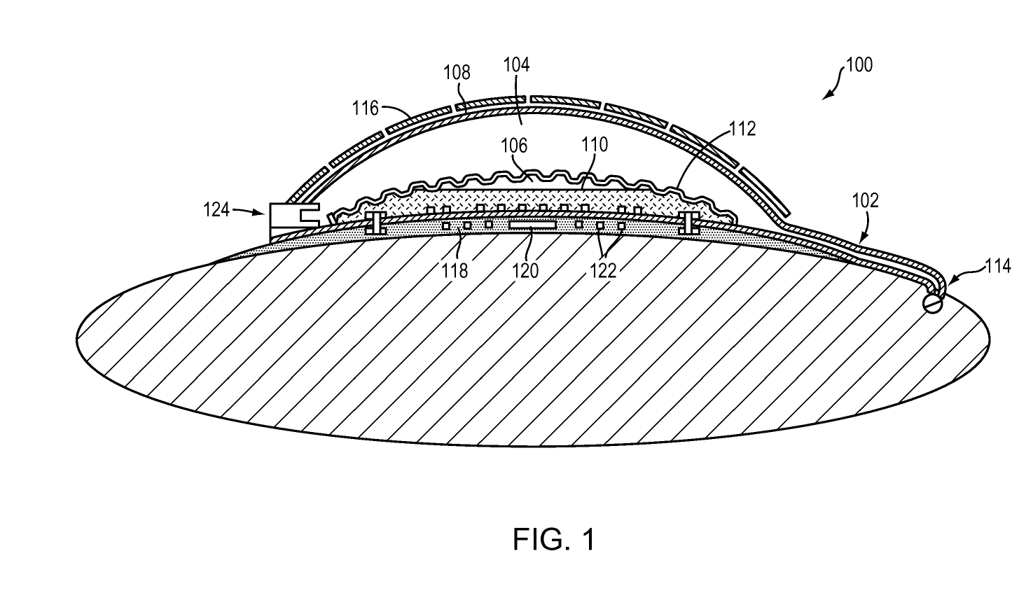

FIG. 1 illustrates an exemplary electrolytically driven drug pump device 100 in accordance herewith (described in detail in U.S. application Ser. Nos. 12/463,251 and 13/632,644, the entire disclosures of which are hereby incorporated by reference). The drug pump device 100 includes a cannula 102 and a pair of chambers 104, 106 bounded by a first envelope 108. The top chamber 104 defines a drug reservoir that contains the drug to be administered in liquid form, and the bottom chamber 106 contains a liquid which, when subjected to electrolysis using electrolysis electrodes 110, evolves a gaseous product. The two chambers are separated by a corrugated diaphragm 112. The cannula 102 connects the top drug chamber 104 with a check valve 114 inserted at the site of administration or anywhere along the fluid path between the drug reservoir and site of administration. The envelope 108 resides within a shaped protective shell 116 made of a flexible material (e.g., a bladder or collapsible chamber) or a relatively rigid biocompatible material (e.g., medical-grade polypropylene). Control circuitry 118, a battery 120, and an induction coil 122 for power and data transmission are embedded between the bottom wall of the electrolyte chamber 106 and the floor of the shell 116. Depending on the complexity of the control functionality it provides, the control circuitry 118 may be implemented, e.g., in the form of analog circuits, digital integrated circuits (such as, e.g., microcontrollers), or programmable logic devices. In some embodiments, the control circuitry 118 includes a microprocessor and associated memory for implementing complex drug-delivery protocols. The drug pump device 100 may also include various sensors (e.g., pressure and flow sensors) for monitoring the status and operation of the various device components, and such data may be logged in the memory for subsequent retrieval and review.

Implantable, refillable drug pump devices need not, of course, have the particular configuration depicted in FIG. 1. Various modifications are possible, including, e.g., devices in which the drug reservoir and pump chamber are arranged side-by-side (rather than one above the other), and/or in which pressure generated in the pump chamber is exerted on the drug reservoir via a piston (rather than by a flexible diaphragm). Furthermore, the pump need not in all embodiments be driven electrolytically, but may exploit, e.g., osmotic or electroosmotic drive mechanisms, or even pressure generated manually.

Importantly for the prolonged use of the drug pump device 100 following implantation, the device 100 includes one or more ports 124 in fluid communication at least with the drug reservoir 104, which permit a refill needle (not shown) to be inserted therethrough.

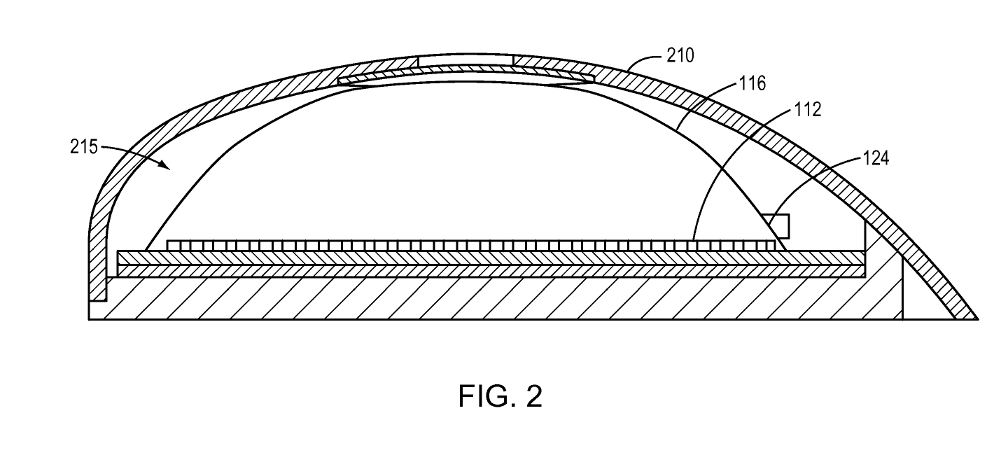

The components illustrated in FIG. 1 may be deployed within a hard outer shell 210, as shown in FIG. 2. The shell 210 may be made of, for example, titanium. The inner shell 116 lies within a second envelope formed by the outer shell 210, creating an enclosed region 215 between the shells 116, 210.

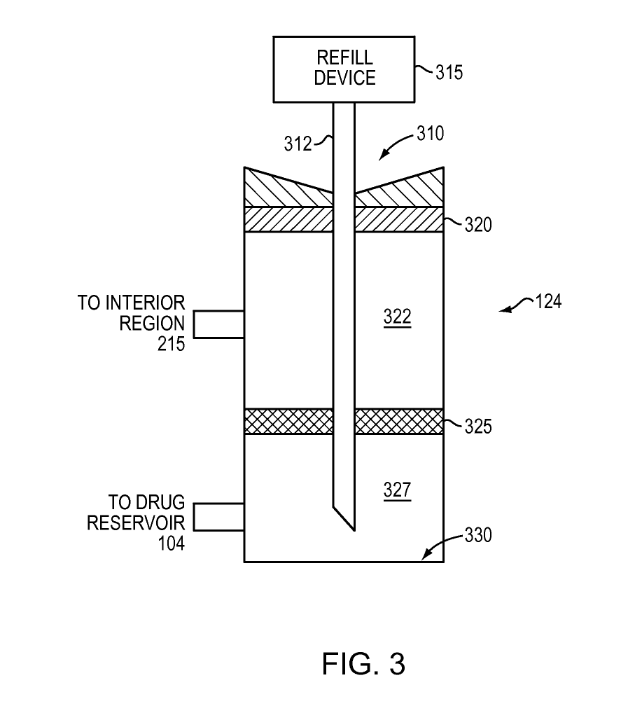

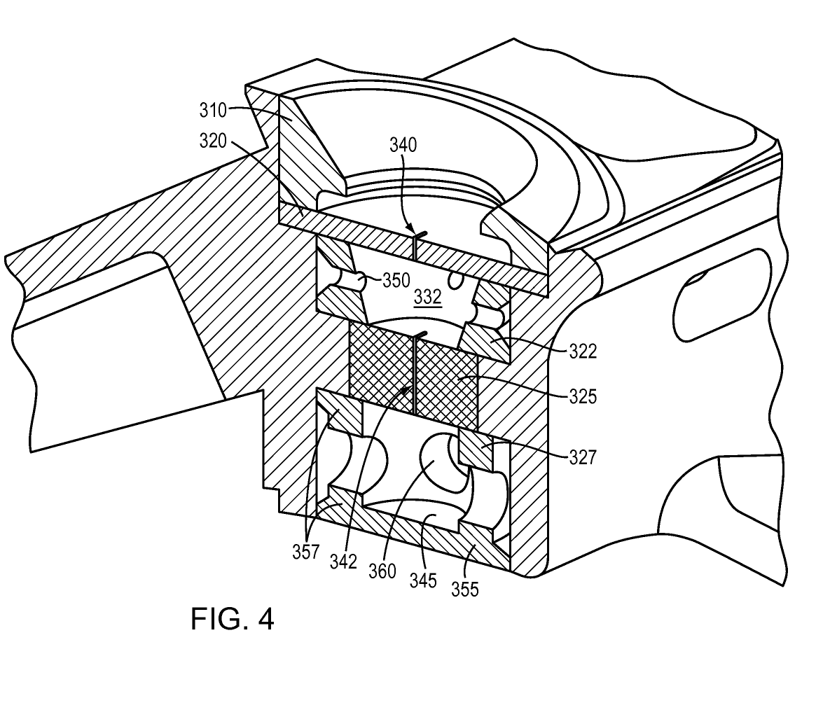

A representative implementation of the inventive venting arrangement is illustrated in FIGS. 3 and 4, which depict the same subject matter in schematic and cutaway form, respectively. It should be understood, however, that various features of the refill port may be used in different combinations or arrangements to meet the orientation, space and functional requirements of the vented device. At the surface of the illustrated refill port 124, an orifice 310 has a conical shape to facilitate convenient and accurate entry of a refill needle 312 extending from a refill device 315. A first septum or membrane 320 underlies the recess and defines a first chamber 322 therebelow. The floor 325 of the first chamber 322 is a second septum 325, which serves as the ceiling of a second chamber 327 therebelow.

The first chamber 322 is in fluid communication with the interior region 215 (see FIG. 2), i.e., the enclosed volume between the outer shell and inner reservoirs of the device and, where gas pressure or vacuum may accumulate without damaging the device or reaching the patient in whom the device is implanted. The second chamber 327 is in fluid communication with a second interior region, which may be the same or, more typically, is different from the interior region 215. The second interior region may, for example, be the drug reservoir 104, in which case the second chamber 327 is used to facilitate refill thereof with medicament or other liquid. That is, liquid expelled from the tip of the needle 312 enters the chamber 327 and is conducted therefrom to the drug reservoir 104.

At least the first septum 320 acts as a check valve operative in either direction—i.e., venting excess gas pressure within the first interior region or permitting ingress of air to relieve a vacuum therein. In various embodiments, one or both septums 320, 325 have a slit 340, 342 that are normally closed due to the elastomeric nature of the septums and, optionally, radial forces of confinement as described in greater detail below.

Both chambers 322, 327 may be defined by a single tubular conduit (with internal features for retaining the septums 320, 325) or may instead be defined separately by individual housings installed within the framework of the pump shell. The latter arrangement allows the chambers to have distinct diameters and interior profiles. Different or varying interior diameters may help guide a refill needle, and different exterior diameters may simplify manufacturing if each housing can only fit into a matching recess within the pump shell. For example, the chamber 322 may be defined by a housing 332 having a conical interior wall in order to maintain a substantially vertical orientation of the needle 312 as it descends into the second chamber 327. In the illustrated embodiment, the refill orifice 310 is surrounded by a ridge with a conical interior profile to guide the refill needle 312, and the conical interior side wall of the first chamber 322 serves the same function. The housing that defines the second chamber 327 is made of a hard material (such as, e.g., titanium, polyurethane, polyethylene, or other metal, plastic or composite) so that the floor 345 thereof acts as a needle stop.

The first septum 320 functions as a check valve that opens once the pressure differential between atmosphere and the interior region 215 reaches the cracking pressure of the septum 320. The check-valve function is typically provided by one or more slits 340 through the septum 320, which also, as noted above, allow the refill needle 312 to pass through the septum 320.

The septum 320 may be made of an elastomeric polymer such as silicone (e.g., polydimethylsiloxane) of a compatible durometer (e.g., 30 to 70 or 80), which allows the septum 320 to be substantially rigid but gives the valve an appropriate cracking pressure suitable for venting while minimizing leaking. Other suitable polymers for the septum 320 include polyurethane, polyethylene, parylene C, or rubber. At least the exterior-facing surface of the septum 320 may have an oleophobic coating thereover to discourage tissue ingrowth and endothilialization. The exterior-facing surface of the septum 320 may have a larger deflection surface to create a disparity in the entering and exiting cracking pressures. The first septum 320 may be preshrunk during the manufacturing process to enhance radial forces tending to increase the cracking pressure.

In the illustrated embodiment, the first chamber 322 is defined by a housing 332 having a spool-like exterior profile, i.e., with terminal flanges and a cylindrical body portion. The venting chamber is in fluid communication with the interior region 215 via one or more radial bores 350 through the body of the chamber housing 332. The bores 350 may be sized and configured to also provide a filtering function. The second septum 325 may be made from any of the materials listed above for the first septum 320, and may be preshrunk to enhance inwardly directed radial forces. The second chamber 327, however, may not serve a venting function, instead merely sealing around the needle 312 to ensure that refill liquid forced through the needle is conducted into the drug reservoir 104 (rather than leaking into the pump via the first chamber or out to the exterior of the pump through the refill port opening).

The second chamber 327 is also defined by a housing 355 having terminal flanges 357 and a cylindrical body portion. The second chamber 327 is in fluid communication with the second interior region via one or more radial bores 360 through the body of the chamber housing 355. Once again the bores 360 may be sized and configured to also provide filtering. As noted, in some embodiments one or both septums have at least one slit, which may span at least the majority of the diameter of the membrane. If more than one slit is made, they will typically intersect at the radial center of the refill port cavity (forming an ‘X’ or asterisk). Alternatively, non-linear slits having, for example, a Z-shape or an S-shape may be employed.

If a piercing refill needle 312 is used, the second septum may not be slit, and ideally is self-healing to substantially recover its sealing properties once the needle is withdrawn. Silicone, for example, is naturally self-healing, but this property is more pronounced in particular formulations well-known to persons of skill in the art. But if blunt needles are to be accommodated, both septums 320, 325 will ordinarily be slit, although the second septum 325 may have a smaller slit 342 and/or greater inwardly radial mounting force to prevent leakage of refill liquid. A multi-lumen needle with exit ports appropriately located along the needle length may be used to assist with the venting and refilling of the device. For example, the exit ports may be located along the length of the needle such that, with the needle tip resting on the floor 345 of the second chamber 327, one exit port is within the second chamber 327 and the other exit port is within the first chamber 322.

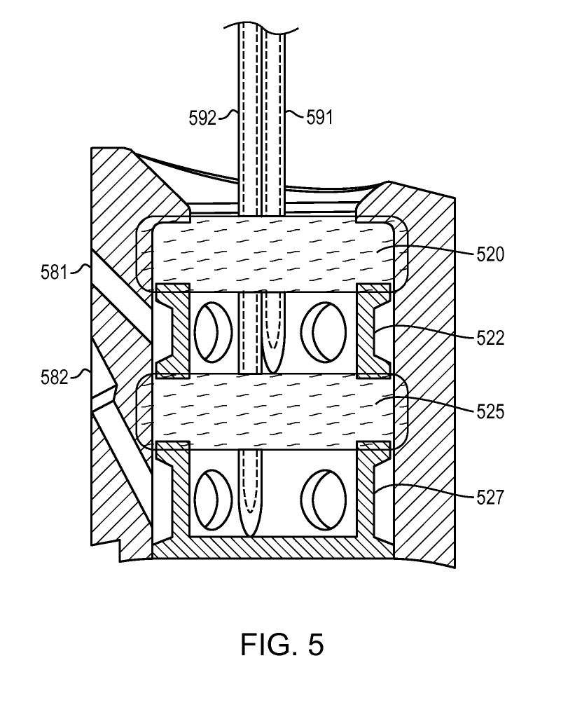

In another embodiment, the first septum does not serve a passive venting function, but instead serves only to equilibrate a pressure or vacuum produced in the interior region 215 as shown in FIG. 5. This embodiment is favorable in use cases where a drug reservoir requires access by a refill/venting needle 312 frequently enough that gas buildup is minimal. With reference to FIG. 5, a representative implementation of this embodiment includes first and second chambers 522, 527 bounded axially by first and second septums 520, 525. A fluid path 581 couples the first chamber 522 to the interior venting region 215 (see FIG. 2), and a fluid path 582 couples the second chamber 527 to the drug reservoir 104 (see FIG. 1). A needle having two lumens, or two adjacent needles 591, 592 as illustrated, penetrate the device so that a first needle outlet is in fluid communication with the first chamber 522 and a second needle outlet is in communication with the second chamber 527. The septums 520, 525 seal securely against the needle(s) 591, 592 and do not provide venting, which instead occurs via the fluid path 581.

The venting fluid path 581 connecting the interior region 215 and the chamber 522 may be a polymer (e.g., silicone or parylene) tube. A selectively permeable membrane structure (which allows gas but not liquid to penetrate) may be integrated into the venting fluid path 581 to ensure that refill liquid forced through the needle is conducted into the drug reservoir 104 rather than leaking into the pump interior region 215 via the first chamber 522. In some embodiments, a check valve or bandpass valve may also be integrated into the venting fluid path 581 to control venting speed to prevent damage to the pump caused by sudden pressure changes. The reservoir fluid path 582 connecting the second interior region (i.e., the drug reservoir) and the chamber 527 housing may be a polymer (e.g., silicone or parylene) tube. The bore size may also be configured to function as a fluid flow-control mechanism to prevent fluid flow rates greater than a safety threshold level (above which the flow could damage the drug reservoir). A bandpass valve (i.e., a valve that allows fluid flow in either direction only when the fluid pressure offset is within a designated range) may be integrated into the reservoir fluid path 582 to prevent over-pressurization or excessive vacuum of the drug reservoir.

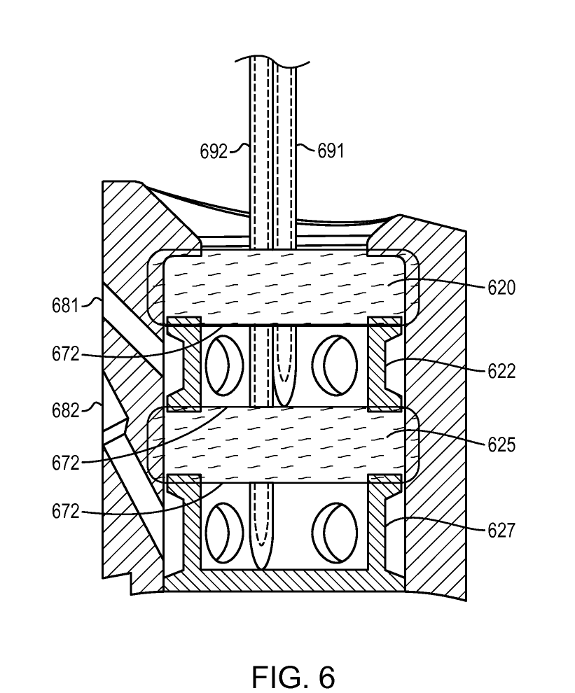

FIGS. 6-9 illustrate various modifications that may be used to minimize septum bulging and deformation while maintaining a low-profile septum. FIG. 6 illustrates an embodiment including first and second chambers 622, 627 bounded axially by first and second septums 620, 625. A fluid path 681 couples the first chamber 622 to the interior venting region 215 (see FIG. 2), and a fluid path 682 couples the second chamber 627 to the drug reservoir 104 (see FIG. 1). A needle having two lumens, or two adjacent needles 691, 692 as illustrated, penetrate the device so that a first needle outlet is in fluid communication with the first chamber 622 and a second needle outlet is in communication with the second chamber 627. A pair of TEFLON layers between 0.001″ to 0.005″ in thickness are applied or bonded to the interior-facing surface of the first septum 620 and both surfaces of the second septum 625. TEFLON provides a low-profile layer that may be adhered to the septum with an epoxy to prevent septum bulging and deformation. Alternatively, the TEFLON layer may be molded into the septum during manufacture.

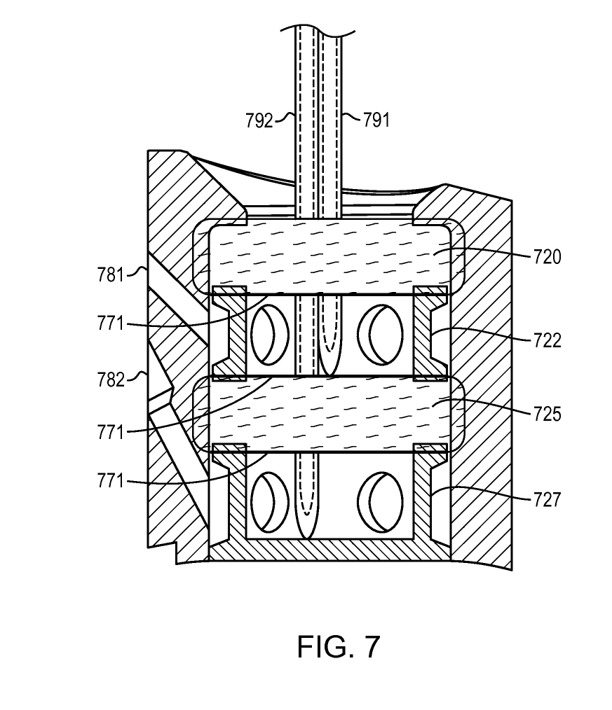

FIG. 7. illustrates the use of a support mesh to provide increased septum integrity. The embodiment shown in FIG. 7 includes first and second chambers 722, 727 bounded axially by first and second septums 720, 725. A fluid path 781 couples the first chamber 722 to the interior venting region 215 (see FIG. 2), and a fluid path 782 couples the second chamber 727 to the drug reservoir 104 (see FIG. 1). A needle having two lumens, or two adjacent needles 791, 792 as illustrated, penetrate the device so that a first needle outlet is in fluid communication with the first chamber 722 and a second needle outlet is in communication with the second chamber 727. The mesh 771 is bonded to the interior-facing surface of the first septum 720 and both surfaces of the second septum 625. The mesh 771 may have a hole size greater than the needle gauge and may be configured is specific patterns (e.g. circles, hexagon, etc.) to prevent the use of larger needles that could permanently damage the septum if they were to travel through the entire septum. Alternatively, the support mesh 771 may be molded into the septum during manufacture.

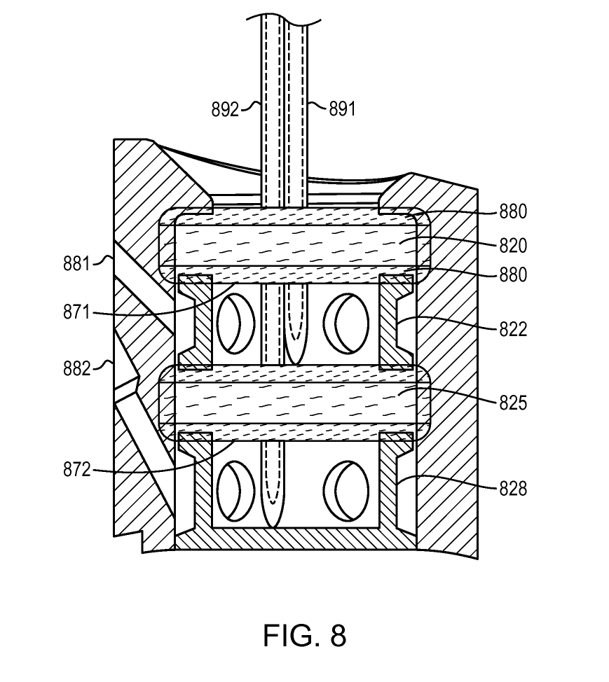

FIG. 8 illustrates use of septums created of two or more silicone materials, one of a high durometer (e.g., 50 to 100) and one of a low durometer (e.g., 10 to 60). The embodiment shown in FIG. 8 includes first and second chambers 822, 827 bounded axially by first and second septums 820, 825. A fluid path 881 couples the first chamber 822 to the interior venting region 215 (see FIG. 2), and a fluid path 882 couples the second chamber 827 to the drug reservoir 104 (see FIG. 1). A needle having two lumens, or two adjacent needles 891, 892 as illustrated, penetrate the device so that a first needle outlet is in fluid communication with the first chamber 822 and a second needle outlet is in communication with the second chamber 827. Each of the septums 820, 825 include a central region having the low durometer and opposed surface regions 880 having the high durometer. In some embodiments, the high- and low-durometer silicones are present in multiple alternating layers, or the higher-durometer silicone may completely encapsulate the lower-durometer interior region. The higher-durometer silicone 880 confers structural rigidity to the septum, minimizes lower molecular weight extractables for better pharmaceutical compatibility, and is suitable for septum surfaces. The lower-durometer silicone has better self-sealing properties but is succeptable to greater bulging and deformation.

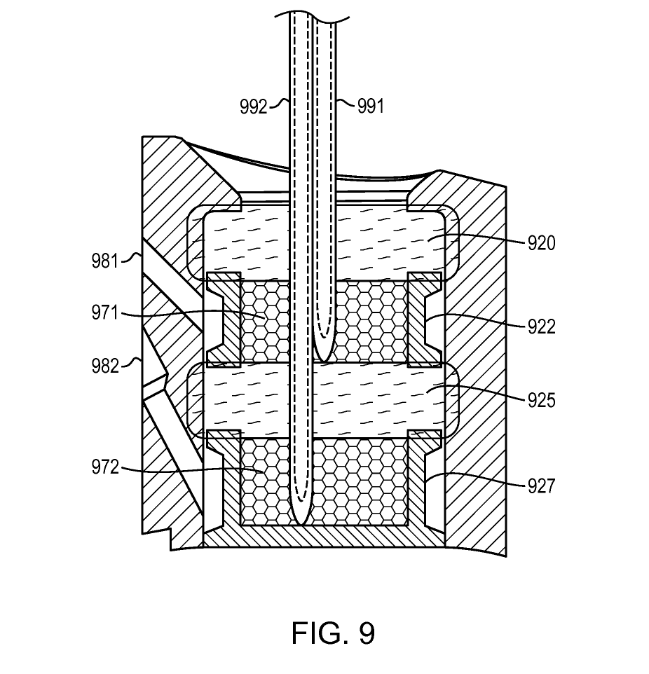

FIG. 9 illustrates the use of an open-celled support structure within the chambers. The embodiment shown in FIG. 9 includes first and second chambers 922, 927 bounded axially by first and second septums 920, 925. A fluid path 981 couples the first chamber 922 to the interior venting region 215 (see FIG. 2), and a fluid path 982 couples the second chamber 927 to the drug reservoir 104 (see FIG. 1). A needle having two lumens, or two adjacent needles 991, 992 as illustrated, penetrate the device so that a first needle outlet is in fluid communication with the first chamber 822 and a second needle outlet is in communication with the second chamber 927. Each chamber 922, 927 is filled with an open-celled support material 971, 972, which preferably fills the entire chamber volume. This material does not significantly inhibit fluid flow but provides structural reinforcement. Suitable open-cell materials include foams such as polyurethane foams.

Any one or more of the various components of the refill port assembly may be manufactured or treated so as to identify the refill port and/or signal proper needle insertion. For example, electrical illumination, chemical illumination, mechanical switches, tactile feedback, magnetic mechanisms, and/or acoustic mechanisms may be employed.

Having described certain embodiments of the invention, it will be apparent to those of ordinary skill in the art that other embodiments incorporating the concepts disclosed herein may be used without departing from the spirit and scope of the invention. For example, various features described with respect to one particular device type and configuration may be implemented in other types of devices and alternative device configurations as well. Accordingly, the described embodiments are to be considered in all respects as only illustrative and not restrictive.

What is claimed is:

1. An implantable device for administering a liquid, the device comprising:
an outer shell defining an interior volume;
within the interior volume, a pump assembly including a reservoir, a gas-driven forcing mechanism and a passage for conducting liquid from the reservoir to an ejection site outside the shell in response to pressure applied by the forcing mechanism; and
a refill port assembly comprising:
an orifice through a surface of the housing for receiving a refill needle;
a first housing defining a first chamber having first and second open ends and fluidly coupled, via at least one bore through the first housing, to a ventable interior portion of the shell;
a second housing defining a second chamber having an open end and fluidly coupled, via at least one bore through the second housing, to the drug reservoir; and
first and second needle-penetrable septums,
wherein the orifice, the first and second housings and the first and second septums are arranged in series with the first septum disposed between, and penetrably sealing, the orifice and the first open end of the first housing, and the second septum disposed between, and penetrably sealing, the second open end of the first housing and the open end of the second housing.

2. The device of claim 1, wherein the first septum is slit to form a check valve facilitating release of pressurized gas or relief of a vacuum within the ventable interior portion of the shell.

3. The device of claim 1, wherein the first septum has a surface comprising an oleophobic coating thereover to discourage tissue ingrowth and endothelialization.

4. The device of claim 1, wherein the first septum comprises of a polymeric material having a durometer ranging from 30 to 80.

5. The device of claim 1, wherein the second septum is made of a self-healing material.

6. The device of claim 1, wherein the bores are sized to function as a filter.

7. The device of claim 1, wherein the second housing comprises a closed end opposite the open end, at least the closed end being made of a needle-impenetrable material.

8. The device of claim 1, wherein the first and second housings and the first and second septums are received within separate recesses within the implantable device.

9. The device of claim 2, wherein the first septum comprises a plurality of slits intersecting at a point.

10. The device of claim 1, wherein the first and second septums further comprise surface TEFLON layers.

11. The device of claim 1, wherein the first and second septums further comprise surface layers of support mesh.

12. The device of claim 1, wherein each of the first and second septums has first and second regions having, respectively, a first and second durometer, the first region including at least a portion of an exterior of the associated septum, the first durometer being higher than the second durometer.

13. The device of claim 1, wherein the first and second chambers are filled with an open-cell material.

14. The device of claim 1, wherein the fluid coupling between the first chamber and the ventable interior portion of the shell comprises a polymer tube having an integrated check valve.

15. The device of claim 1, wherein the fluid coupling between the second chamber and the drug reservoir comprises a polymer tube having an integrated check valve.
